(19) United States
(12) Patent Application Publication
(10) Pub. No.: US 2025/0035540 A1
(43) Pub. Date: Jan. 30, 2025

(54) DETECTION OF A SOLUTE INJECTED INTO A GAS PHASE OPTICAL SPECTROMETER

(71) Applicant: Aerodyne Research, Inc., Billerica, MA (US)

(72) Inventors: Joseph R. Roscioli, Chelmsford, MA (US); Elizabeth M. Lunny, North Billerica, MA (US); Joanne H. Shorter, Lexington, MA (US); Scott C. Herndon, Littleton, MA (US)

(21) Appl. No.: 18/226,494

(22) Filed: Jul. 26, 2023

(51) Int. Cl. G01N 21/31; G01N 33/24

(52) U.S. Cl. CPC G01N 21/31; G01N 33/24

(57) ABSTRACT

In various embodiments, improved techniques are provided for detecting and/or mapping a solute in a medium by injecting the solute as droplets into a heated, evacuated absorption cell (e.g., a multipass absorption cell) of a gas phase optical spectrometer (e.g., an infrared laser absorption spectrometer). The techniques may be applicable to a wide range of solutes in soil (e.g., hydroxylamine, nitrite, nitrate, etc.) involved in subsurface nitrogen cycling processes, as well as other solutes involved in other types of processes in soil or other mediums.

### STATEMENT REGARDING FEDERALLY SPONSORED RESEARCH AND DEVELOPMENT

This invention was made with U.S. Government support under Grant No. 2021-33530-34614, awarded by the National Institute of Food and Agriculture. The U.S. Government has certain rights in this invention.

### BACKGROUND

### Technical Field

The present disclosure relates generally to detecting and/or mapping solutes, and more particularly to tools and techniques for detection of a solute in a medium, such as soil, water, biological media, plant material, etc.

### Background Information

Understanding and controlling the nitrogen cycle in soil is a fundamental tool of modern agriculture. The nitrogen cycle is a biogeochemical cycle by which nitrogen is converted into multiple chemical forms as it circulates among ecosystems. The nitrogen cycle in soil can beneficially increase plant-available nutrients, but can also detrimentally lead to losses, such as greenhouse gas emissions and leaching. Understanding and controlling the nitrogen cycle in soil is therefore important not only to improve plant health and yield, but also to minimize ecological impacts. However, existing tools and techniques for exploring the nitrogen cycle in soil face a number of shortcomings.

Various molecules and ions involved in subsurface nitrogen cycling processes are often present in soil as solutes in soil water. To explore the nitrogen cycle in soil, it is typically desirable to accurately detect, and in some cases map, such solutes with high spatial and temporal resolution. One solute that may be desirable to detect and map is hydroxylamine (NH₂OH). Hydroxylamine is an inorganic highly reactive compound that is an intermediate or side metabolite for different nitrogen cycle microorganisms and plays a role in nitric oxide (NO) and nitrous oxide (N₂O) emissions. Other examples of solutes involved in the nitrogen cycle in soil that it may be desirable to detect and/or map include nitrite (NO₂) and nitrate (NO₃⁻), among others. However, hydroxylamine, nitrate, nitrate, and other solutes have traditionally been very difficult to detect and/or map. Measurement usually has required labor-intensive, destructive soil sampling and offline measurement using gas chromatography, colorimetry and/or mass spectrometry. Results from these processes usually suffered from low temporal and spatial resolution. Further, in the case of hydroxylamine and other “sticky” molecules, their gas-phase measurement was complicated by their tendency to temporarily bind to surfaces inside of instruments (e.g., due to their large dipole moments and/or hydrophilic properties). As such, rapid, accurate measurement was challenging.

Accordingly, there is a need for improved techniques for detecting and/or mapping solutes in a medium.

### SUMMARY

In various embodiments, improved techniques are provided for detecting and/or mapping a solute in a medium by injecting the solute as droplets into a heated, evacuated absorption cell (e.g., a multipass absorption cell) of a gas phase optical spectrometer (e.g., an infrared laser absorption spectrometer). The techniques may be applicable to a wide range of solutes in soil (e.g., hydroxylamine, nitrite, nitrate, etc.) involved in subsurface nitrogen cycling processes, as well as other solutes involved in other types of processes in soil or other mediums.

In various embodiments, a solute is extracted (e.g., in aqueous solution) by a number of probes (e.g., microdialysis (MD) probes) arranged in a medium (e.g., soil) at different locations. Solute (dialysate) from each probe is multiplexed (e.g., by a selector valve) and loaded as a solute sample (e.g., by a sample injector into a sample loop). Each solute sample is successively injected as droplets of liquid into a heated, evacuated absorption cell (e.g., a multipass absorption cell) of a gas phase optical spectrometer (e.g., an infrared laser absorption spectrometer). The droplets of the injected solute sample rapidly evaporate in the heated, low pressure environment of the absorption cell to produce a gas phase trapped sample. Light (e.g., laser light) is directed to the trapped sample in the absorption cell, and an absorption spectrum obtained from which concentration may be determined using well-known gas phase optical spectrometry (e.g., infrared laser absorption spectrometry) techniques. By cycling through solute samples from the probes (e.g., MD probes) at the different locations over a measurement period, a temporal and spatial map of a solute may be created with high resolution in both domains.

Some solutes (e.g., hydroxylamine, etc.) may be directly detected in their vaporized form. Other solutes (e.g., nitrite, nitrate, etc.) may be detected in a converted form that is more amenable the volatilization needed for gas phase measurement (e.g., nitrous acid (HONO) for nitrite, nitric acid (HNO₃) for nitrate, etc.), resulting from acid/base addition or electrochemical conversion. In some embodiments, conversion to the form amenable to volatilization may be promoted by sample preparation (e.g., performed by a sample preparation unit disposed between the sample injector and the absorption cell).

Such techniques may provide a number of advantages over traditional processes For example, rather than attempt to draw interstitial gas to the instrument, solution is brought to the instrument which may have a greater amount of the solute in it than is available in interstitial gas. Further, challenges resulting from the “stickiness” of certain samples (e.g., hydroxylamine) may be avoided, as they are passed through the majority of the instrument as a solute, rather than in their more “sticky” gaseous form.

In one example embodiment, a method is provided for detecting and/or mapping a solute in a medium. Solute is extracted from one or more probes that are each arranged at a respective location in the medium. A solute sample is loaded from the extracted solute from each of the one or more probes. Each solute sample is injected as droplets of liquid into a heated, evacuated absorption cell of a gas phase optical spectrometer, wherein the droplets evaporate internal to the absorption cell due to its heated, low pressure environment to produce a gas phase trapped sample in the absorption cell. Gas phase optical spectrometry is used to determine concentration from each trapped sample in the absorption cell. An indication of detected solute is output based on the determined concentration from each trapped sample or a map of detected solute is output based on the determined concentration from each trapped sample.

In another example embodiment, an instrument is provided for detecting and/or mapping a solute in a medium. The instrument includes one or more probes configured to extract solute from respective locations in the medium and includes a sample injector configured to successively load a predetermined amount of solute into a sample loop from each of the one or more probes and inject each solute sample as droplets of liquid. The instrument further includes gas phase optical spectrometer having an absorption cell configured to receive the injected droplets of each solute sample, wherein the absorption cell is further configured to cause the droplets to evaporate internal to the absorption cell to produce a gas phase trapped sample in the absorption cell. The instrument further includes control electronics configured to execute software that controls the gas phase optical spectrometer to determine concentration from each trapped sample in the absorption cell and to output an indication of detected solute based on the determined concentration from each trapped sample or a map of detected solute based on the determined concentration from each trapped sample.

It should be understood that a variety of additional features and embodiments may be implemented other than those discussed in this Summary. This Summary is intended simply as a brief introduction to the reader for the further description that follows and does not indicate or imply that the features and embodiments mentioned herein cover all aspects of the disclosure or are necessary or essential parts of the disclosure.

### BRIEF DESCRIPTION OF THE DRAWINGS

The description refers to the accompanying drawings of example embodiments, of which:

FIG. 1 is a diagram of an example instrument for detecting and/or mapping a solute in a medium that injects the solute as droplets into an absorption cell of a gas phase optical spectrometer;

FIG. 2 is a graph illustrating an example of detection of hydroxylamine that may be produced by operation of the example instrument of FIG. 1;

FIG. 3 is a graph illustrating an example of detection of nitrate using nitric acid as a proxy;

FIG. 4 is a graph illustrating an example of detection of nitrite using nitrous acid as a proxy;

FIGS. 5A-5C are an enlarged diagram showing an evacuated absorption cell, with call-outs illustrating evaporation induced volatilization of species;

FIG. 6A is a graph illustrating an example of conversion by acid addition of nitrate to gaseous nitric acid at varying levels of acidity;

FIG. 6B is a graph illustrating dependence on temperature of conversion of nitrate to gaseous nitric acid, showing various temperatures; and

FIG. 7 is a flow diagram of an example sequence of steps for detecting and/or mapping a solute in a medium that involves injecting the solute as droplets into a absorption cell.

### DETAILED DESCRIPTION

The following detailed description describes example embodiments. Any references to items in the singular should be understood to include items in the plural, and vice versa, unless explicitly stated otherwise or otherwise clear from the context. Grammatical conjunctions are generally intended to express any and all disjunctive and conjunctive combinations of conjoined clauses, sentences, words, and the like, unless otherwise stated or clear from the context. For example, the term “or” should generally be understood to mean “and/or.”

Any recitation of ranges of values are not intended to be limiting, are provided as example only, and are not intended to constitute a limitation on the scope of the described embodiments. Further, any recitation of ranges should be interpreted as referring individually to any and all values falling within the range, unless otherwise indicated, and each separate value within such a range should be treated as if it were individually recited. Terms of approximation such as “about,” “approximately,” “substantially” or the like, should be construed as referring to an allowance for deviation that is appreciated by one of ordinary skill in the art to still permit satisfactory operation for the corresponding use, function, purpose, or the like. No language in the description should be construed as indicating that an element is a necessary or essential aspect of the disclosure.

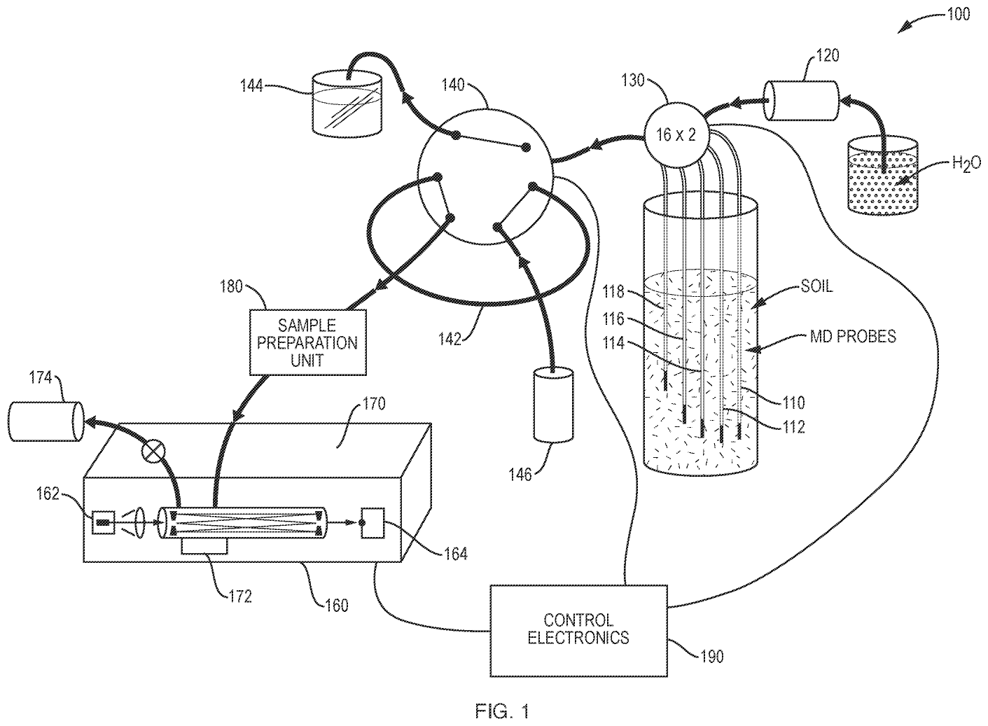

FIG. 1 is a diagram of an example instrument 100 for detecting and/or mapping a solute in a medium that injects the solute as droplets into an absorption cell of a gas phase optical spectrometer. The solute may take a number of different forms. In some embodiments, the solute is a molecule or ion involved in subsurface nitrogen cycling processes in aqueous solution (e.g., soil water) and the medium is soil. For example, the solute may be hydroxylamine, nitrite, nitrate, or another molecule or ion in aqueous solution that plays a role in the nitrogen cycle in soil. It should be understood, however, that the solute need not be limited to those that play a role in the nitrogen cycle. In other embodiments, the solute may be another type of molecule or ion in solution, for example, acetate (C₂H₃O₂⁻), carbonate (CO₃⁻²), formate (CHO₂⁻) ammonium (H₄N⁺), cyanide (CN⁻), sulfate (O₄S⁻²), phosphate (O₄P⁻³), halides (e.g., fluoride (F⁻), chloride (Cl⁻), bromide (Br⁻), iodide (I⁻), etc.) in water or another type of solution, involved in other types or processes in soil or other types of mediums (e.g., water (a river, ocean, ice melt, etc.), biological medium (blood, plasma, urine, etc.), plant material (a stem, xylem/phloem, fruit, etc.), and the like). Further, it should be understood that the instrument 100 may not be limited to detection and/or mapping of a single solute, and multiple (e.g., 2) solutes may be simultaneously or alternately examined.

The instrument includes a number of probes 110-118 arranged in the medium (e.g., soil) at different locations. In one embodiment, 16 probes are employed, however it should be understood that a smaller or larger number may be employed, including in some cases a single probe. The probes 110-118 may be MD probes that include a hollow shaft with a semipermeable membrane at the tip. In operation, each MD probe is perfused with water at a low flow rate (e.g., a flow rate of 0.1-10 μL/min.) by a pump 120 to collect solute (dialysate) from the medium (e.g., soil). In embodiments where there are multiple MD probes, an electrically-actuated selector valve 130 (e.g., a 16×2 selector valve) may be caused by software of control electronics 190 (e.g., a computer, microcontroller, or other electronic device) to cycle delivery of solute from the probes 110-118 to a sample injector 140.

The sample injector 140 successively loads and injects solute samples of a predetermined size (e.g., 1-25 μL, for example 10 μL). In one embodiment, the sample injector 140 is a 6 port electrically-actuated sample injector valve that operates under the direction of the software of the control electronics 190. With the sample injector valve 140 in a first position (a sample loading position), the valve 140 causes solute to flow into a first end of a sample loop 142 that is coupled on its opposing end to a waste/dump receptacle 144, and for flush air from a flush air pump 146 to be injected to clear downstream components. The sample loop may be sized to accommodate the predetermined microliter-scale volume of solute sample (e.g., sized to accommodate 1-25 μL of solute sample, for example 10 μL of solute sample). When the sample injector valve 140 is in the second position (a sample injection position), the valve 140 causes the solute sample to be injected under pressure from the flush air, and for any excess solute sample being received from the selector valve 130 to be directed to the waste/dump receptacle 144. By cycling the sample injector valve between the two positions, the software of the control electronics 190 may cause a succession of solute samples from the probes 110-118 to be provided.

Each solute sample may pass through an optional sample preparation unit 180 just before injection, the operations of which are discussed further below. Each solute sample is injected into a heated, evacuated absorption cell (e.g., multipass absorption cell) 160 of a gas phase optical spectrometer (e.g., an infrared laser absorption spectrometer) 170. The absorption cell 160 may be heated by a heater 172. Depending on the configuration, the heater 172 may produce different levels of elevated temperature (i.e., temperature above the ambient environment, for example, a temperature between 25-150° C.) in the cell. The absorption cell 160 may be evacuated by a vacuum pump 174. Depending on the configuration, differing levels of partial vacuum may be achieved.

In traditional instruments, a gaseous sample is typically supplied to a absorption cell 160. However, breaking from this convention, the instrument 100 injects the solute sample as droplets of liquid directly into the heated, evacuated absorption cell 160. The droplets of liquid rapidly evaporate upon entry into the heated, low-pressure environment of the absorption cell 160. Evaporation of the droplets and heat drives volatilization. The result may be a 10-50 Torr (e.g., an approximately 20 Torr) gaseous trapped sample inside the cell.

Producing such a sample internal to the absorption cell 160 may yield a number of advantages over traditional processes. The solute sample from which the trapped sample is produced may include a greater amount of solute in it than is available in interstitial gas. Likewise, challenges resulting from the “stickiness” of certain samples may be avoided as they are passed all the way to the absorption cell 160 as a solute, rather than in their more reactive gaseous form.

The gas phase optical spectrometer 170 may be of any of a number of types. In one embodiment, the gas phase optical spectrometer 170 is an infrared laser absorption spectrometer, or more specifically a tunable infrared laser direct absorption spectroscopy (TILDAS) trace gas analyzer. In one configuration the TILDAS trace gas analyzer has an optical path length of 76 meters and a cell volume of 0.5 liters. In operation, one or more lasers (e.g., quantum-cascade lasers (QCLs)) 162 under control of the software of control electronics 190 direct laser light to the absorption cell 160, where it repeatedly traverses the length of the cell reflecting off a series of broadband mirrors to achieve the path length. Each laser 162 may be tuned to emit light near a spectral region suitable for detection of a particular species, and the use of multiple lasers may facilitate detection of multiple gases. In operation, light exits the absorption cell 160 and is received by one or more optical detectors (e.g., mercury cadmium telluride (MCT) detectors) 164, which provide signals describing spectral lines to the software of control electronics 190.

It should be understood that in other embodiments, the gas phase optical spectrometer 170 may be another type of spectrometer, and that such other type of spectrometer may use other types of light sources, absorption cells, detectors, and the like. For example, the gas phase optical spectrometer 170 may alternatively be a Fourier transform infrared (FTIR) spectrometer, an ultraviolet-visible (UV/VIS) absorption spectrometer, a non-dispersive infrared (NDIR) spectrometer, a cavity attenuated phase shift (CAPS) spectrometer, a cavity ringdown spectrometer, etc.

The gas phase optical spectrometer 170, under direction of the software of the control electronics 190, performs a measurement of the concentration (e.g., particle number concentration) of each trapped sample in the absorption cell 160. The measurements may be performed using the well-known Beer-Lambert law, which provides that light transmitted is a function of the frequency for an isolated line of the light output of the laser as a function of laser frequency, the optical path length, molecular number density, mixing ratio (concentration) and absorption line strength. Further details of the construction of an example TILDAS trace gas analyzer and use of the Beer-Lambert law for measurement of concentration of a sample may be found in J. B. McManus et at., “Recent Progress in Laser-Based Trace Gas Instruments: Performance and Noise Analysis”, Appl. Phys. B (2015) 199:203-218, the contents of which are incorporated by reference herein in their entirety.

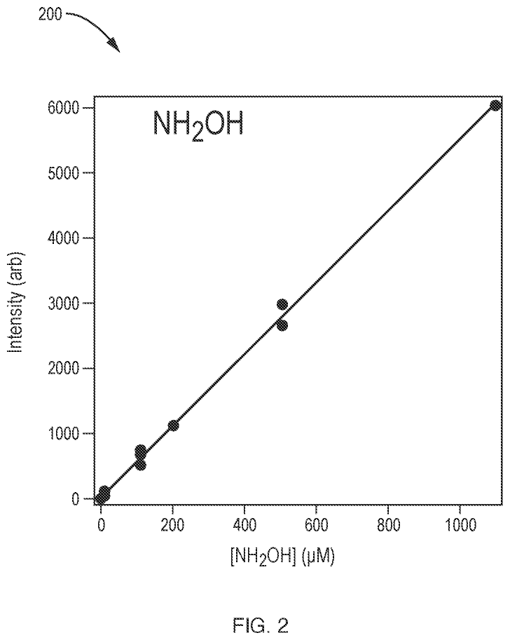

Some solutes may be directly detected in the absorption cell 160 in their vaporized form. For example, hydroxylamine may be directly detected upon vaporization. A laser 162 may be tuned for detection in a spectral region near 1354 cm⁻¹, near 3600 cm⁻¹, or another spectral region for detection of hydroxylamine. FIG. 2 is a graph 200 illustrating an example of detection of hydroxylamine that may be produced by operation of the example instrument 100 of FIG. 1. Here, concentration of hydroxylamine is plotted against intensity of an isolated line.

Other solutes may be detected in a converted form that is more amenable to volatilization needed for gas phase measurement, as a result of acid/base addition or electrochemical conversion. For example, nitrite may be detected as nitrous acid (acid addition), nitrate may be detected as nitric acid (acid addition), acetate may be detected as acetic acid (CH₃COOH) (acid addition), carbonate may be detected as carbonic acid (H₂CO₃) (acid addition) or carbon dioxide (CO₂) (base addition), formate may be detected as formic acid (CH₂O₂) (acid addition), ammonium may be detected as ammonia (NH₃) (base addition), cyanide may be detected as hydrogen cyanide (HCN) (acid addition), sulfate may be detected as sulfur dioxide (SO₂) (electrochemical reduction) or hydrogen sulfide (H₂S) (electrochemical reduction), phosphate may be detected as phosphine (PH₃) (electrochemical reduction), halides maybe detected as dihalides (XY) (electrochemical reduction), etc.

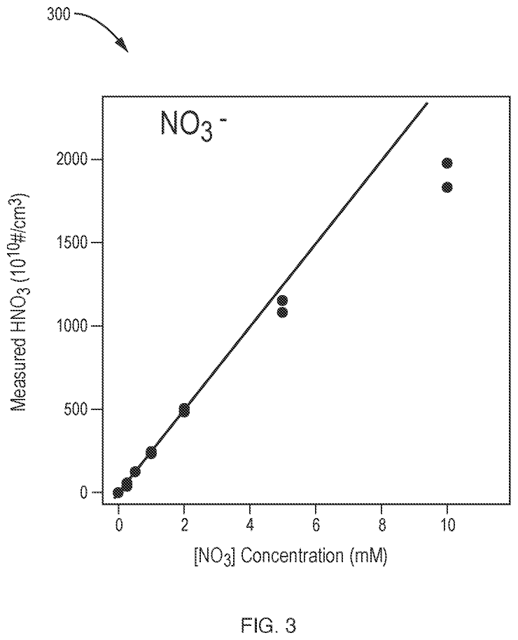

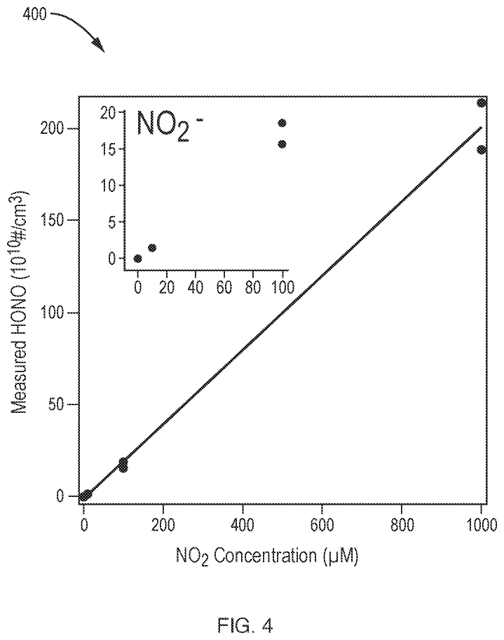

FIG. 3 is a graph 300 illustrating an example of detection of nitrate using nitric acid as a proxy. Here, concentration of nitrate is plotted against measured nitric acid. Similarly, FIG. 4 is a graph 400 illustrating an example of detection of nitrite using nitrous acid as a proxy. Here, concentration of nitrite is plotted against measured nitrous acid.

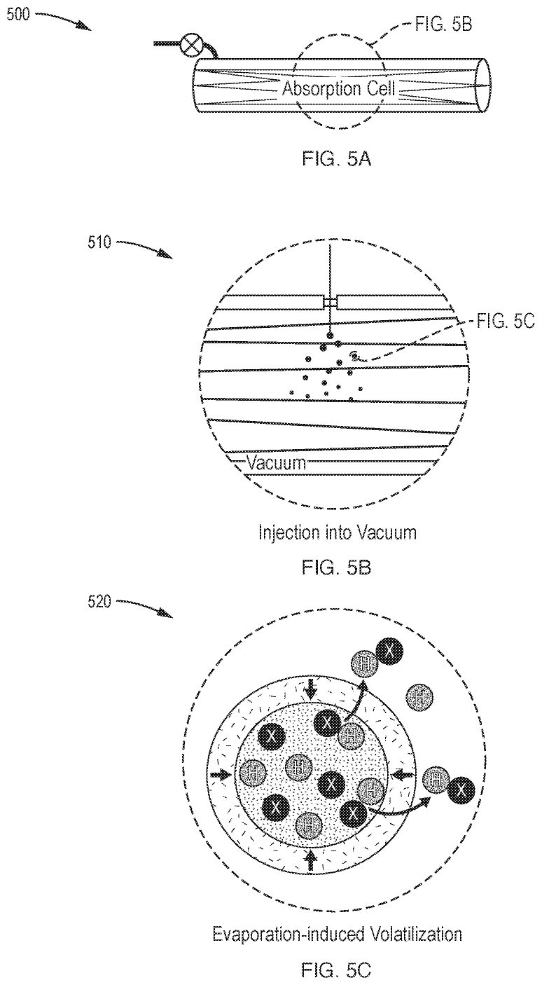

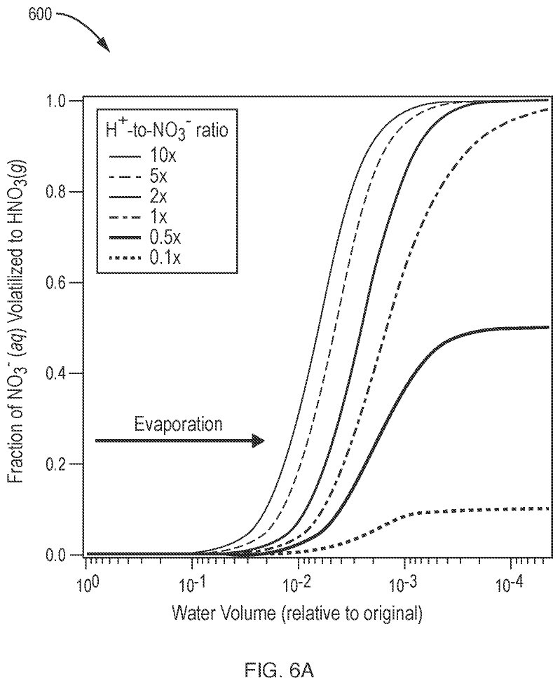

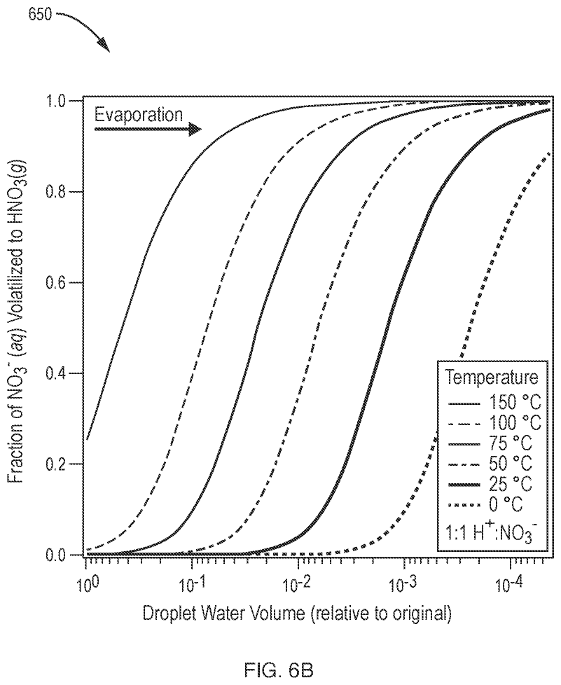

Conversion to a form amenable to volatilization and gas phase measurement is promoted by evaporation under vacuum. FIGS. 5A-5C are an enlarged diagram 500 showing an evacuated absorption cell (e.g., an evacuated multipass absorption cell) 160, with call-outs 510, 520 illustrating evaporation induced volatilization of species. Call-out 510 illustrates injection of droplets of liquid solute sample into the evacuated cell 160, where they rapidly evaporate. Call-out 520 illustrates evaporation-induced volatilization forming a trapped sample of HX. FIG. 6A is a graph 600 illustrating an example of conversion by acid addition of nitrate to gaseous nitric acid amenable to volatilization and gas phase measurement at varying levels of acidity. As can be observed, as the volume of the liquid solute sample decreases due to droplet evaporation, the fraction of nitrate volatized to nitric acid increases. Temperature aids volatilization by both shifting equilibrium towards the converted form and driving enhanced partial pressure. FIG. 6B is a graph 650 illustrating dependence on temperature of conversion of nitrate to gaseous nitric acid, showing various temperatures.

As mentioned above, in some embodiments an optional sample preparation unit 180 may be provided between the sample injector 140 and the absorption cell 160 that may promote conversion to a form amenable to volatilization and gas phase measurement. The sample preparation unit 180 may chemically alter the solute to increase its volatility before injection into the cell 160. The sample preparation unit 180 may include a mixing unit for mixing in sources of additional hydrogen ions (H⁺), hydroxide ions (OH⁻), or other additives to the solute sample to promote conversion. For example, referring back to FIG. 6, it can be seen in the graph 600 that the efficiency of conversion of nitrate to nitric acid depends on the availability of hydrogen ions. By mixing in sources of additional hydrogen ions, the sample preparation unit 180 may make the solute sample more volatile and drive conversion in the absorption cell 160. In some cases, the sample preparation unit 180 may use a microfluidic or electrolytic reactor to render the solute sample more volatile before it is injected into the absorption cell 160.

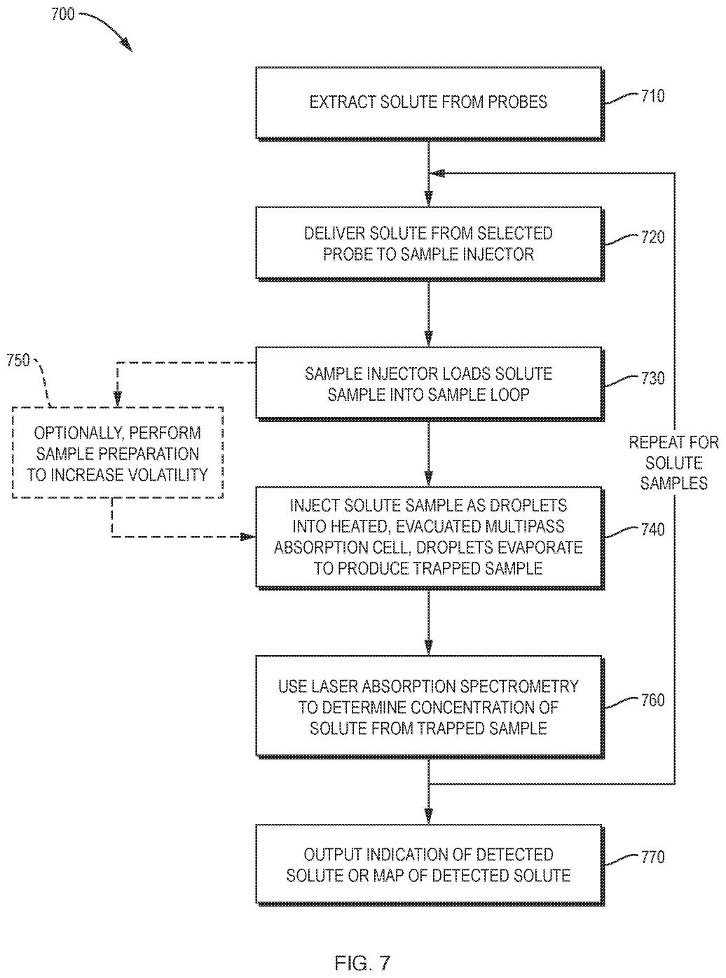

FIG. 7 is a flow diagram of an example sequence of steps 700 for detecting and/or mapping a solute in a medium that involves injecting the solute as droplets into an absorption cell of a gas phase optical spectrometer. The steps 700 may serve to recap and summarize some of the above discussed operations. At step 710, solute is extracted from one or more (e.g., 16) probes (e.g., MD probes) 110-118 arranged at respective locations in a medium (e.g., soil). Each probe may be perfused with water to collect solute (dialysate) from the medium (e.g., soil). At step 720, an electrically-actuated selector valve (e.g., a 16×2 selector valve) 130 under control of software of control electronics 190 selects delivery of solute from one of the probes 110-118 to a sample injector 140. At step 730, an electrically-actuated sample injector valve 140 under control of software of the control electronics 190 loads a solute sample (e.g., into a sample loop 142) having a predetermined size (e.g., 1-25 μL, for example 10 μL).

At step 740, the sample injector valve 140 under control of software of the control electronics 190 injects the solute sample as droplets of liquid into a heated (e.g., 25-150° C.), evacuated (e.g., 10-50 Torr) absorption cell (e.g., multipass absorption cell) 160 of a gas phase optical spectrometer (e.g., an infrared laser absorption spectrometer, such as a TILDAS trace gas analyzer) 170. The droplets evaporate internal to the absorption cell due to its heated, low pressure environment to produce a gas phase trapped sample in the absorption cell 160. At optional step 750, which may occur just before the solute sample reaches the absorption cell, a sample preparation unit 180 disposed between the sample injector 140 and the absorption cell 160 increases volatility by mixing an additive into the solute sample (e.g., additional hydrogen ion, hydroxide ions (OH), etc.).

At step 760, software of the control electronics 190 controls the gas phase optical spectrometer 170 to determine concentration from the trapped sample in the absorption cell 160. Gas phase optical spectrometry may be used to directly detect a vaporized form of the solute or to detect a gaseous converted form of the solute resulting from acid/base addition or electrochemical conversion. Steps 720-760 may be repeated to cycle through solute samples from the probes (e.g., MD probes) 110-118 at the different locations over a measurement period, resulting in a set of measurements.

Thereafter, at step 770, the software of the control electronics 190 outputs (e.g., displays in a user interface on a display screen, saves to a file or other data store, transmits over a network, etc.) an indication (e.g., a table, graph, or other representation) of the detected solute or a map of the detected solute, based on the set of measurements. In the case of a map, the map may be a temporal and spatial map that shows changes in solute concentrations at different locations over the measurement period.

In summary, the above description describes improved techniques for detecting and/or mapping a solute in a medium by injecting the solute as droplets into a heated, evacuated absorption cell of a gas phase optical spectrometer. It should be understood that various adaptations, modifications and extensions may be made to suit various design requirements and parameters. The ordering of any method steps discussed above may be changed to suit various applications or requirements. Absent an explicit indication to the contrary, the order of steps described above may be modified such that a subsequent step occurs before a preceding step, or in parallel to such step. Above all, it should be understood that the above descriptions are meant to be taken only by way of example. Numerous variations, additions, omissions, and other modifications will be apparent to one of ordinary skill in the art, and such variations, additions, omissions, and other modifications should be considered within the scope of this disclosure. Thus, while example embodiments have been shown and described, it will be apparent to those skilled in the art that changes, and modifications, may be made therein without departing from the spirit and scope of this disclosure.

What is claimed is:

1. A method for detecting and/or mapping a solute in a medium, comprising:
extracting solute from one or more probes that are each arranged at a respective location in the medium;
loading a solute sample from the extracted solute from each of the one or more probes;
injecting each solute sample as droplets of liquid into a heated, evacuated absorption cell of a gas phase optical spectrometer, wherein the droplets evaporate internal to the absorption cell due to its heated, low pressure environment to produce a gas phase trapped sample in the absorption cell;
using gas phase optical spectrometry to determine concentration from each trapped sample in the absorption cell; and
outputting an indication of detected solute based on the determined concentration from each trapped sample or a map of detected solute based on the determined concentration from each trapped sample.

2. The method of claim 1, wherein the one or more probes are microdialysis (MD) probes that are perfused with water, and the solute is a dialysate from the MD probes.

3. The method of claim 1, wherein the medium is soil.

4. The method of claim 1, wherein the one or more probes are a plurality of probes, and the method further comprises:
multiplexing solute from each of the plurality of probes to a sample injector that successively produces solute samples that are injected into the absorption cell.

5. The method of claim 1, wherein the gas phase optical spectrometry directly detects a vaporized form of the solute.

6. The method of claim 1, wherein the solute is hydroxylamine (NH₂OH).

7. The method of claim 1, wherein the gas phase optical spectrometry detects a converted form of the solute resulting from acid/base addition or electrochemical conversion.

8. The method of claim 7, wherein the solute is nitrite (NO₂⁻) and the converted form is nitrous acid (HONO), the solute is nitrate (NO₃⁻) and the converted form is nitric acid (HNO₃), the solute is acetate (C₂H₃O₂⁻) and the converted form is acetic acid (CH₃COOH), the solute is carbonate (CO₃⁻²) and the converted form is carbonic acid (H₂CO₃) or carbon dioxide (CO₂), the solute is formate (CHO₂⁻) and the converted form is formic acid (CH₂O₂), the solute is ammonium (H₄N⁺) and the converted form is ammonia (NH₃), the solute is cyanide (CN⁻) and the converted form is hydrogen cyanide (HCN), the solute is sulfate (O₄S⁻²), and the converted form is sulfur dioxide (SO₂), the solute is phosphate (O₄P⁻³) and the converted form is phosphine (PH₃), or the solute is a halide and the converted form is a dihalide.

9. The method of claim 7, wherein the converted form is a more volatile form and the method further comprise:
promoting conversion of the solute in the solute sample to the more volatile form by mixing an additive into the solute sample before injection into the absorption cell.

10. The method of claim 9, wherein the additive provides additional hydrogen ions (H⁺) or hydroxide ions (OH⁻).

11. The method of claim 1, wherein the extracting, loading, injecting, and using are repeated to cycle through solute samples from the probes at the different locations over a measurement period, and the outputting outputs a temporal and spatial map.

12. An instrument for detecting and/or mapping a solute in a medium, comprising:
one or more probes configured to extract solute from respective locations in the medium;
a sample injector configured to successively load a predetermined amount of solute into a sample loop from each of the one or more probes and inject each solute sample as droplets of liquid;
a gas phase optical spectrometer having an absorption cell configured to receive the injected droplets of each solute sample, wherein the absorption cell is further configured to cause the droplets to evaporate internal to the absorption cell to produce a gas phase trapped sample in the absorption cell; and
control electronics configured to execute software that controls the gas phase optical spectrometer to determine concentration from each trapped sample in the absorption cell and to output an indication of detected solute based on the determined concentration from each trapped sample or a map of detected solute based on the determined concentration from each trapped sample.

13. The instrument of claim 12, further comprising:
a heater configured to heat the absorption cell to an elevated temperature to induce volatilization of species.

14. The instrument of claim 12, further comprising:
a vacuum pump configured to evacuate the absorption cell to a partial vacuum to promote evaporation of the trapped sample and induce volatilization of species.

15. The instrument of claim 12, wherein the one or more probes are microdialysis (MD) probes that are configured to be perfused with water, and the solute is a dialysate from the MD probes.

16. The instrument of claim 12, wherein the medium is soil.

17. The instrument of claim 12, wherein the one or more probes are a plurality of probes, and the instrument further comprises:
a selector valve configured to multiplex solute from each of the plurality of probes to the sample injector, wherein the software of the control electronics is configured to control the selector valve and sample injector to successively produces solute samples that are injected into the absorption cell over a measurement period.

18. The instrument of claim 12, wherein the gas phase optical spectrometer is controlled to determine concentration by directly detecting a vaporized form of the solute from each trapped sample.

19. The instrument of claim 12, wherein the gas phase optical spectrometer is controlled to determine concentration by detecting a converted form of the solute resulting from acid/base addition or electrochemical conversion.

20. The instrument of claim 1, wherein the solute is hydroxylamine (NH₂OH), nitrite (NO₂⁻), nitrate (NO₃⁻), acetate (C₂H₃O₂⁻), carbonate (CO₃⁻²), formate (CHO₂⁻), ammonium (H₄N⁺), cyanide (CN⁻), sulfate (O₄S⁻²), phosphate (O₄P⁻³) or a halide.
